(19) United States
(12) Patent Application Publication
(10) Pub. No.: US 2006/0062781 A1
(43) Pub. Date: Mar. 23, 2006

(54) Specific ab1'-antibodies against tumour-associated antigen ca 125

(75) Inventors: Harald Schlebusch, Starnberg (DE); Uwe Wagner, Marburg (DE)

Correspondence Address: MORRIS MANNING & MARTIN LLP, 1600 ATLANTA FINANCIAL CENTER, 3343 PEACHTREE ROAD, NE, ATLANTA, GA, 30326-1044, US

(73) Assignee: CELLCONTROL BIOMEDICAL Laboratories AG, 82319 Starnberg (DE)

(21) Appl. No.: 10/507,046

(22) PCT Filed: Mar. 7, 2003
(86) PCT No.: PCT/EP03/02362

§ 371 Date: Sep. 9, 2005

(30) Foreign Application Priority Data
Mar. 8, 2002 (DE) 102 10 239.2

(51) Int. Cl. A61K 39/395; C12N 5/06; C07K 16/42

(52) U.S. Cl. 424/131.1; 435/327; 530/387.2

(57) ABSTRACT

The present invention concerns specific anti-anti-idiotypic antibodies which react with anti-idiotypic antibodies which represent an internal image of the antigen CA125 and which also bind to the antigen itself i.e. are specific for the tumour-associated antigen CA125. In particular the present invention concerns a CA125-specific anti-anti-idiotypic antibody which reacts with the anti-idiotypic antibody ACA125. In addition the present invention concerns pharmaceutical compositions containing these anti-anti-idiotypic antibodies for treating tumours that express CA125 and in particular ovarian carcinomas.

The present invention concerns specific anti-anti-idiotypic antibodies which react with anti-idiotypic antibodies which represent an internal image of the antigen CA125 and which also bind to the antigen itself. In particular the present invention concerns anti-anti-idiotypic antibodies which react with the anti-idiotypic ACA125 which represents an internal image of the tumour-associated antigen CA125. In addition the present invention concerns pharmaceutical compositions containing these anti-anti-idiotypic antibodies for treating tumours that express CA125 and in particular ovarian carcinomas.

Immunotherapies for treating tumour diseases have for several years been the subject matter of numerous research studies. Although tumour-associated antigens are known for various types of tumour, a regression of the tumour by vaccination with such an antigen has only been observed in rare cases. Thus for example attempts have been made to treat ovarian carcinomas by vaccination with the tumour-associated antigen CA125. However, a tumour rejection by the immune system was unsuccessful. It is assumed that the close relationship between the tumour-associated antigen CA 125 and autoantigens is responsible for the lack of anti-tumour immunity.

A promising approach for overcoming the immunological tolerance of the organism to the tumour-associated antigen CA125 is based on the idiotypic network hypothesis using anti-idiotypic antibodies which represent an internal image of the antigen CA125. This requires anti-idiotypic antibodies whose antigen binding region represents a close copy of the CA125 antigen determinant and thus can functionally imitate the CA125 antigen. The anti-idiotypic antibody ACA125 which is produced by the hybridoma 3D5 (DSM ACC 2120) imitates the tumour-associated antigen CA125 and is used for immunization against CA125-expressing tumours (EP 0 700 305 B1 and U.S. Pat. No. 5,858,361).

The object of the present invention was to provide anti-anti-idiotypic antibodies against anti-idiotypic antibodies which represent an internal image of the antigen CA125 and especially against the anti-idiotypic antibody ACA125 which react with the tumour-associated antigen CA125 itself and are thus able to mediate an antibody-dependent cellular cytotoxicity (ADCC) against CA125-expressing tumour cells.

This object is solved according to the invention in that anti-anti-idiotypic anti-CA125 antibodies were discovered which can trigger an ADCC against CA125-expressing tumour cells.

Hence the present invention concerns anti-anti-idiotypic antibodies which
(i) react with an anti-idiotypic antibody which represents an internal image of the antigen CA125,
(ii) are specific for the tumour-associated antigen CA125 and react with this antigen, and
(iii) mediate an antibody-dependent cellular cytotoxicity against CA125-expressing tumour cells.

The antibodies according to the invention are so-called Ab1′ antibodies. Starting with a tumour-associated antigen CA125, the antibodies which are induced by this antigen and are specific for this antigen are named Ab1 antibodies. Previously known Ab1 antibodies are of murine origin and cannot mediate an ADCC reaction. The Ab1 antibodies whose formation is induced by immunization with a CA125 antigen have specific variable sections for antigen recognition. Hence, Ab1 antibodies can specifically bind to CA125. However, the variable sections also contain sequences that are themselves effective as an antigen. These sequences are called idiotypic determinants, are themselves immunogen and can thus induce the formation of anti-idiotypic antibodies, so-called Ab2 antibodies. An example of such an Ab2 antibody is ACA125. Some of the Ab2 antibodies are able to imitate the three-dimensional structure (internal image) of the original antigen i.e. of CA125. Hence these Ab2 antibodies can in turn be used as an antigen to induce antibodies. In particular contact with an anti-idiotypic Ab2 antibody can induce anti-anti-idiotypic antibodies, so-called Ab3 antibodies in the patient by means of idiotype-positive B cells. Whereas Ab3 antibodies bind to the anti-idiotypic antibody Ab2, such antibodies do not bind to the corresponding antigen, in this case CA125. The invention concerns so-called Ab1′ antibodies which are induced by an anti-idiotypic antibody Ab2. The Ab1′ antibodies according to the invention can bind to the anti-idiotypic antibody Ab2 as well as to the original tumour antigen CA125. These antibodies according to the invention are also able to mediate an antibody-dependent cellular cytotoxicity against CA125-expressing tumour cells. Furthermore the Ab1′ antibodies according to the invention are in particular antibodies of human origin.

In a preferred embodiment of the present invention the anti-anti-idiotypic antibody is an antibody which reacts with the monoclonal anti-idiotypic antibody ACA125 produced by the hybridoma 3D5 (DSM ACC 2120), but in particular with the antigen CA125.

The anti-anti-idiotypic antibody according to the invention can be present as a polyclonal or monoclonal antibody.

The polyclonal anti-anti-idiotypic antibodies according to the invention can be produced by any conventional method known to a person skilled in the art for producing polyclonal antibodies. The polyclonal anti-anti-idiotypic antibodies are preferably produced as a polyclonal immune response by vaccination with an anti-idiotypic antibody. The polyclonal anti-anti-idiotypic antibodies are particularly preferably produced by vaccination with the monoclonal anti-idiotypic antibody ACA125.

The monoclonal anti-anti-idiotypic antibodies according to the invention can be produced by conventional methods known to a person skilled in the art for producing monoclonal antibodies. For example the hybridoma technique is suitable using human cells. Hence another subject matter of the present invention is a human hybridoma cell line which produces the monoclonal anti-anti-idiotypic antibody according to the invention. When using murine hybridoma cells to produce monoclonal antibodies it is only possible to obtain antibodies which although having the binding capability of the complete anti-anti-idiotypic antibody, but are not able in this form i.e. as murine monoclonal antibodies to mediate an ADCC reaction.

Such murine antibodies or fragments thereof in particular Fab or F(ab)₂ fragments must then be coupled to human Fc parts in order to obtain the anti-anti-idiotypic antibodies according to the invention.

In another preferred embodiment of the present invention the anti-anti-idiotypic antibody can be produced recombinantly.

Another subject matter of the present invention concerns a fragment of an anti-anti-idiotypic antibody according to the invention which has the binding capability of the complete anti-anti-idiotypic antibody and at the same time mediates an ADCC reaction. The fragment according to the invention preferably comprises at least a Fab or F(ab)₂ fragment and a human Fc part. For this purpose it is also possible to couple or fuse a fragment comprising at least a Fab or F(ab)₂ fragment which has the binding capability of a complete anti-anti-idiotypic antibody with a human Fc part which mediates an ADCC reaction. The fragment can preferably react with an anti-idiotypic antibody which represents an internal image of the antigen CA125, is specific for the tumour-associated antigen CA125 and can mediate an antibody-dependent cellular cytotoxicity against CA125-expressing tumour cells. Furthermore the fragment according to the invention can be produced by conventional methods known to a person skilled in the art such as proteolysis, limited proteolysis, recombinant expression etc.

Yet a further subject matter of the present invention is a pharmaceutical composition comprising anti-anti-idiotypic antibodies according to the invention or fragments thereof according to the invention. The pharmaceutical composition according to the invention can additionally contain common pharmaceutical carriers and adjuvants, where necessary.

The pharmaceutical composition according to the invention can be present in a form that can be administered topically, parenterally, intravenously, intramuscularly, subcutaneously or transdermally and can be produced with the aid of conventional processes known to a person skilled in the art. The pharmaceutical composition according to the invention is preferably produced in the form of solutions or suspensions.

The pharmaceutical composition according to the invention is used for the treatment and/or prophylaxis of CA125-expressing tumours. The pharmaceutical composition according to the invention is preferably used for the treatment and/or prophylaxis of ovarian carcinomas.

Thereby, the pharmaceutical composition according to the invention is administered to a patient with a tumour disease in an amount that is sufficient to treat the corresponding CA125-expressing tumour. The amount of pharmaceutical composition to be administered depends on several factors such as the type of administration (injection, infusion etc.), the type and extent of the tumour disease and the age, weight and general condition of the patient and can be easily determined by a person skilled in the field of tumour diseases taking into account the above-mentioned factors.

The pharmaceutical composition according to the invention is administered topically, parenterally, intravenously, intramuscularly, subcutaneously or transdermally. The pharmaceutical composition is preferably administered as an injection and/or infusion. In individual cases the pharmaceutical composition can also be specifically injected into body cavities or by means of a catheter into the blood vessels of the tumour region or organ in which the tumour is located.

The following figures and the following example are intended to illustrate the invention in more detail.

### FIGURES

FIG. 1 shows a group of patients in which it was possible to generate a polyclonal immune response in the form of an anti-anti-idiotypic antibody which reacts with the anti-idiotypic antibody ACA125 (so-called AB3 antibody). Induction of polyclonal antibodies which specifically react with the antigen CA125 (so-called Ab1′ antibodies) and ADCC response.

FIG. 2 shows the detection of an ADCC response to CA125-positive cells (OAW-42) versus CA125-negative cells (SKOV-3) in 14 of 26 female patients.

FIG. 3 shows the detection of CA125-specific Ab1′ antibodies in ADCC-positive (A and C) and ADCC-negative (B and D) female patients. A and B: Binding of preantisera and postantisera (1:20) to CA125-positive (OAW-42) versus CA125-negative cells (SKOV-3). C and D: detection of free Ab1′ antibodies and Ab1′ immunocomplexes in postantisera (1:50) using isolated CA125 antigen (ELISA). Preantisera exhibited no reactivity with the CA125 antigen.

### EXAMPLE

Introduction

As part of a clinical phase I/II study ovarian carcinoma patients were immunized with the anti-idiotypic antibody ACA125 (Ab2) which functionally imitates the tumour-associated antigen CA125. The induction of specific anti-anti-idiotypic Ab3 antibodies against ACA125 (Ab2) is considered as a surrogate marker for an immune reaction and has a positive effect on the survival of the patients. The aim of the present study was to further characterize the Ab3 response and to show to what extent the anti-anti-idiotypic Ab3 antibodies can mediate an antibody-dependent cellular cytotoxicity (ADCC) against CA125-expressing tumour cells.

Procedure

The induction of CA125-specific Ab1′ antibodies was evaluated in a group of 26 Ab3-positive female patients (Ab3>10,000 arbU/ml) with the aid of different assay formats. In order to detect the binding to membrane-bound CA125 antigen, CA125-positive (OAW-42) and CA125-negative (SKOV-3) ovarian carcinoma cells were incubated with preantisera and postantisera of the patients (1:20) and analysed by flow cytometry after staining with FITC-conjugated anti-human IgG.

In addition the ability of free Ab1′ antibodies and of Ab1′ antibodies that are bound to circulating CA125 (complexes) in serum (1:50) to react with isolated CA125 antigen was examined (ELISA). For this CA125-coated microtitre plates were incubated with preantisera and postantisera and bound Ab 1′ was detected by a two-step detection using ACA125 (Ab2) and HRP-labelled anti-mouse IgG (Fc-specific). The Ab1′ immune complexes were detected in an analogous manner after dissociation of the complexes by acid and heat treatment of the sera (1:50).

In addition the ADCC-response mediated by Ab1′ was checked on the basis of the lysis of CA125-positive and negative cells by PBLs from healthy subjects (effector:target ratio 25:1) after addition of heat-inactivated preantisera and postantisera (1:20) of the patients (method: LDH release assay).

Results

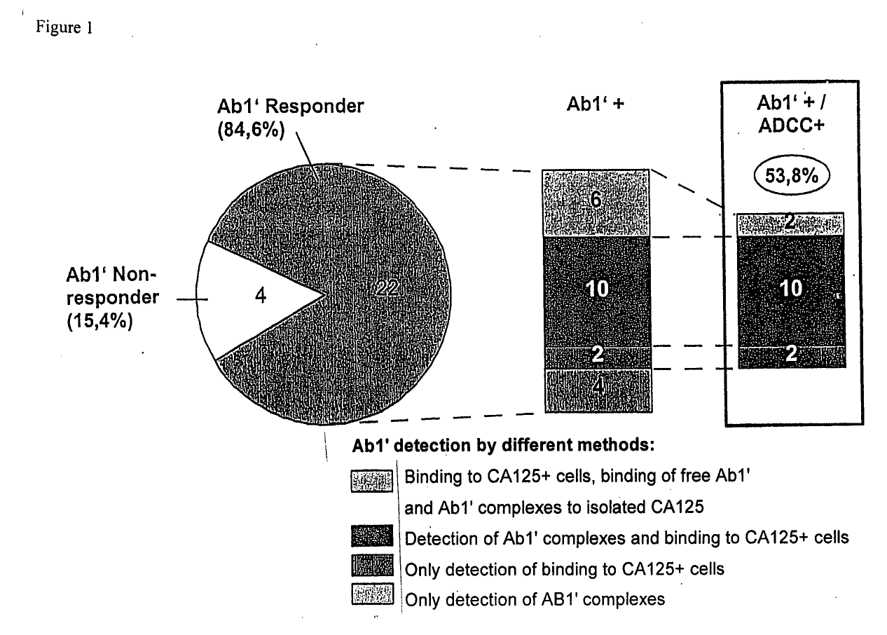

The formation of specific Ab1′ antibodies was detected by various detection methods in 22 of 26 female patients who reacted with a positive Ab3 immune response after immunization with the anti-idiotypic antibody ACA125. However, one discrepancy was observed between the reactivity with isolated CA125 antigen and CA125 on ovarian carcinoma cells since 18 patients had specific antibodies to CA125-positive cells, whereas a reactivity with isolated CA125 was only detected in 6 cases. Different sensitivities of the assay formats and loss of epitopes by the purification of CA125 are possible explanations for this phenomenon. However, after dissociation of Ab 1′-antigen complexes, a reactivity with isolated antigen was detected in 20 patients (FIG. 1).

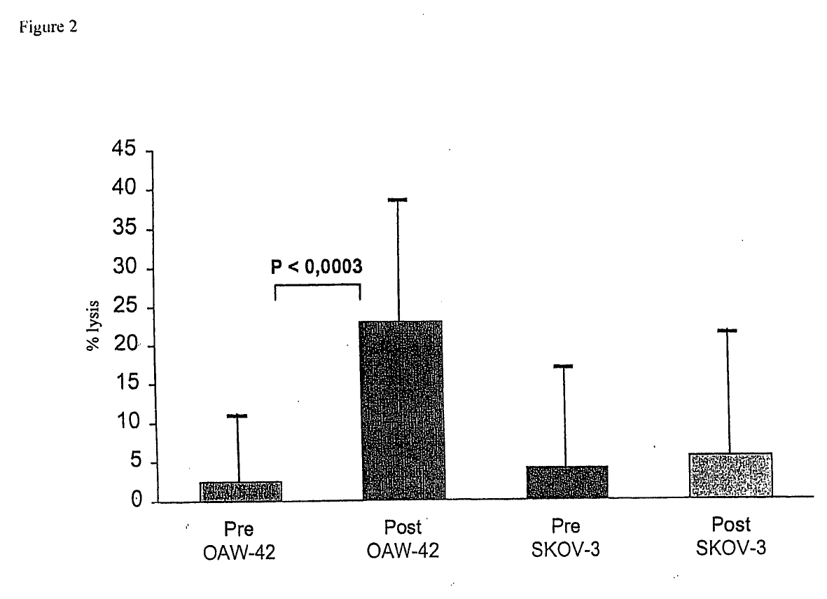

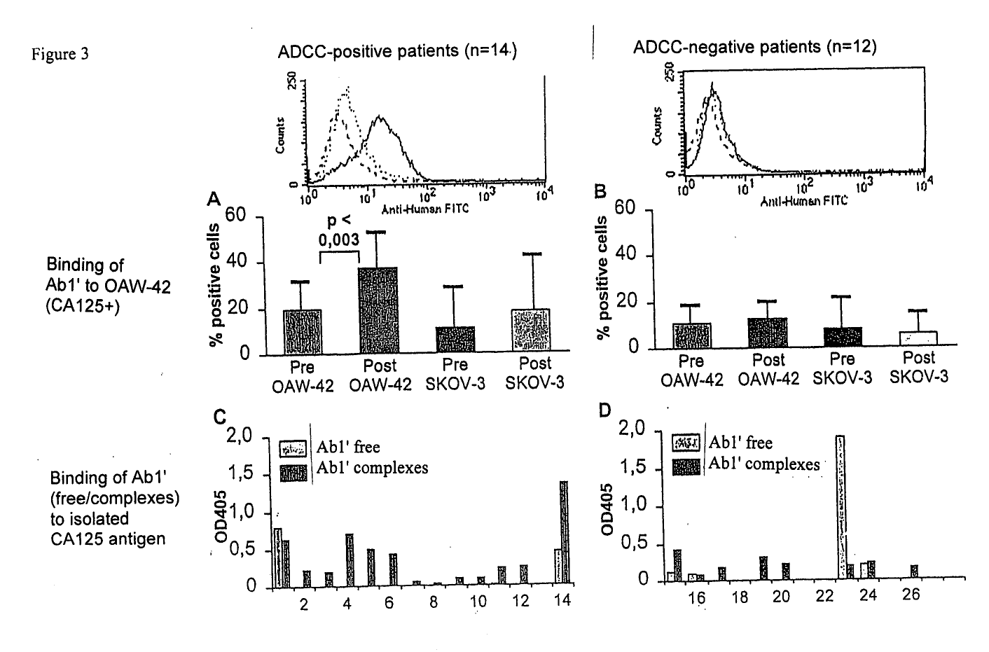

An ADCC to CA125-positive cells after addition of postantiserum was observed in 14 of the 26 female patients (10.8 to 50% lysis) (FIG. 2). These were patients whose Ab1′ antibodies bind specifically to CA125-positive cells (FIG. 3A). In contrast, in the 12 ADCC-negative patients only a weak binding to CA125-positive cells was found in 4 cases (FIG. 3B). There was therefore no direct correlation between the reactivity of free Ab1′ antibodies and/or Ab1′ complexes with isolated CA125 antigen and the ADCC response since these were equally detectable in ADCC-negative and ADCC-positive patients (FIGS. 3C and D).

### CONCLUSION

The anti-idiotype vaccine ACA125 is able to overcome the immunological tolerance to CA125 since CA125-specific Ab1′ antibodies were detected in 85% of the Ab3-positive patients. The results of the present study show that Ab1′ antibodies which bind to CA125-positive cells can mediate a specific ADCC response which thus represents a possible cytotoxic mechanism of the anti-idiotype vaccination. However, the formation of immunocomplexes between Ab1′ antibodies and circulating CA125 appears in some cases to prevent the induction of a cytotoxic reaction in the form of an ADCC.

1. Anti-anti-idiotypic antibody,
characterized in that it
(i) reacts with an anti-idiotypic antibody which represents an internal image of the antigen CA125,
(ii) is specific for the tumour-associated antigen CA125 and reacts with this antigen, and
(iii) mediates an antibody-dependent cellular cytotoxicity against CA125-expressing tumour cells.

2. Anti-anti-idiotypic antibody as claimed in claim 1,
characterized in that
it reacts with the monoclonal anti-idiotypic antibody ACA125 produced by the hybridoma 3D5 (DSM ACC2120).

3. Anti-anti-idiotypic antibody as claimed in claim 1 or claim 2,
characterized in that
it is produced as a result of a polyclonal immune response by vaccination with an anti-idiotypic antibody which represents an internal image of the antigen CA125.

4. Anti-anti-idiotypic antibody as claimed in claim 1 or claim 2,
characterized in that
it is produced recombinantly.

5. Anti-anti-idiotypic antibody as claimed in claim 1 or claim 2,
characterized in that
it is produced by hybridoma cells as a monoclonal antibody.

6. Human hybridoma cell line which produces the monoclonal anti-anti-idiotypic antibody as claimed in claim 5.

7. Fragment of an anti-anti-idiotypic antibody as claimed in any one of the claims 1 to 5,
characterized in that
it has the binding specificity of the anti-anti-idiotypic antibody.

8. Fragment as claimed in claim 7,
characterized in that
it comprises a Fab or F(ab)₂ fragment of the anti-anti-idiotypic antibody optionally coupled to a human Fc part.

9. Pharmaceutical composition comprising an anti-anti-idiotypic antibody as claimed in any one of the claims 1 to 5 or a fragment as claimed in claim 7 or claim 8.

10. Pharmaceutical composition as claimed in claim 8,
characterized in that
it additionally contains common pharmaceutical carriers and adjuvants.

11. Pharmaceutical composition as claimed in claim 9 or claim 10 for the treatment and/or prophylaxis of CA1125-expressing tumours.

12. Use of an anti-anti-idiotypic antibody as claimed in any one of the claims 1 to 5 or a fragment as claimed in claim 7 or claim 8 for the treatment and/or prophylaxis of CA125-expressing tumours.
